(19) United States
(12) Patent Application Publication
(10) Pub. No.: US 2024/0000994 A1
(43) Pub. Date: Jan. 4, 2024

(54) PEPTIDE NANOGELS FOR ACCELERATED WOUND HEALING

(71) Applicant: KING ABDULLAH UNIVERSITY OF SCIENCE AND TECHNOLOGY, Thuwal (SA)

(72) Inventors: Charlotte A.E. HAUSER, Thuwal (SA); Hepi Hari SUSAPTO, Thuwal (SA)

(21) Appl. No.: 18/218,174

(22) Filed: Jul. 5, 2023

Related U.S. Application Data
(63) Continuation-in-part of application No. 17/401,542, filed on Aug. 13, 2021.
(60) Provisional application No. 63/067,962, filed on Aug. 20, 2020.
(60) Provisional application No. 63/067,913, filed on Aug. 20, 2020.
(60) Provisional application No. 63/523,658, filed on Jun. 28, 2023.
(60) Provisional application No. 63/358,563, filed on Jul. 6, 2022.

(51) Int. Cl. A61L 26/00

(52) U.S. Cl. CPC A61L 26/0047; A61L 26/008; A61L 26/0066

(57) ABSTRACT

Described are nanogels suitable for scaffolds for encapsulating human dermal fibroblasts for non-healing chronic wounds. Peptide nanogels Ac-IVZK-NH₂ and Ac-IVFK-NH₂ are selected and produce silver nanoparticles in situ within the nanogels to assess their efficacy on micropigs with full-thickness excision wounds. The in situ generation of the silver nanoparticles is done solely through UV irradiation and no reducing agent is used. Application of the peptide nanogels on full thickness micropig wounds demonstrate that the scaffolds are biocompatible and do not trigger wound inflammation. This suggests that scaffolds are safe for topical application. A comparison of the effect of both nanogels even without the addition of the silver nanoparticles, reveals that the scaffold itself has a high potential as an antibacterial agent, which may suppress both the inflammatory reaction and the activity of proteases. The effect on wound closure of the peptide nanogels is comparable to standard care hydrogels.

### CROSS-REFERENCE TO RELATED APPLICATIONS

This application claims benefit of priority of U.S. patent application Ser. No. 17/401,542 entitled, “SCAFFOLDS FROM SELF-ASSEMBLING TETRAPEPTIDES SUPPORT 3D SPREADING, OSTEOGENIC DIFFERENTIATION AND ANGIOGENESIS OF MESENCHYMAL STEM CELLS” filed Aug. 13, 2021, which in turn claims priority to U.S. Provisional Patent Application No. 63/067,913, entitled “PEPTIDE COMPOUND WITH REPETITIVE SEQUENCE” filed Aug. 20, 2020 and to U.S. Provisional Patent Application No. 63/067,962, entitled “TETRAMERIC SELF-ASSEMBLING PEPTIDES SUPPORT 3D SPREADING AND OSTEOGENIC DIFFERENTIATION OF MESENCHYMAL STEM CELLS” filed Aug. 20, 2020 of which the present application is a continuation-in-part application. This application also claims priority to U.S. Provisional Patent Application No. 63/358,563, entitled “SELF-ASSEMBLING PEPTIDES FOR DRUG DELIVERY APPLICATIONS” filed Jul. 6, 2022, and to U.S. Provisional Patent Application No. 63/525,658, entitled “DESIGN AND DEVELOPMENT OF PEPTIDE-MODIFIED ANTINEOPLASTIC DRUGS FOR TARGETING BREAST CANCER CELLS” filed Jun. 28, 2023. The entire contents and disclosures of these patent applications are incorporated herein by reference in their entirety.

This application refers to “EVALUATION OF PEPTIDE NANOGELS FOR ACCELERATED WOUND HEALING IN NORMAL MICROPIGS,” in Frontiers in Nanoscience and Nanotechnology journal published on Nov. 19, 2018. The entire contents and disclosures of these patent applications are incorporated herein by reference.

### BACKGROUND

### Field of the Invention

The present disclosure relates generally to non-healing chronic wounds and, more particularly, to dressings that promote the healing of chronic wounds.

### Background of the Invention

Non-healing chronic wounds are severe complications, which can often eventually lead to amputations. Impairment of wound healing in patients with complicated chronic wounds as well as in patients with diabetic ulcers affects the quality of life of millions of people and requires a high cost to cure. Wound healing is a complicated process that relies on communication between different cell types, growth factors, and extracellular matrix molecules. As such, there is a clear clinical need for dressings that promote the healing of chronic wounds.

### SUMMARY

According to first broad aspect, the present disclosure provides a peptide capable of forming a gel by self-assembly comprising at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

According to a second broad aspect, the present disclosure provides a hydrogel or organogel comprises a peptide, wherein the peptide comprises at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

According to a third broad aspect, the present disclosure provides a method of preparing a hydrogel or organogel, the method comprising dissolving a peptide in an aqueous solution or an organic solution, respectively, wherein the peptide comprises at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

According to a fourth broad aspect, the present disclosure provides a cell or tissue graft comprising a hydrogel, wherein the hydrogel comprises a peptide capable of forming a gel by self-assembly comprising at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

According to a fifth broad aspect, the present disclosure provides a wound dressing comprising a hydrogel, wherein the hydrogel comprises at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

According to a sixth broad aspect, the present disclosure provides a surgical implant comprising a peptide scaffold, wherein the peptide scaffold is formed by a peptide capable of forming a gel by self-assembly comprising: at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

According to a seventh broad aspect, the present disclosure provides a pharmaceutical composition for treating a wound comprises a peptide capable of forming a gel by self-assembly comprising: at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

According to an eighth broad aspect, the present disclosure provides a kit comprising a first container with a peptide and a second container with an aqueous or organic solution, wherein the peptide comprises at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

According to a ninth broad aspect, the present disclosure provides a bioimaging device comprises a peptide capable of forming a gel by self-assembly comprising: at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

According to a tenth broad aspect, the present disclosure provides a 2D or 3D cell culture substrate comprises a peptide capable of forming a gel by self-assembly comprising: at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

According to an eleventh broad aspect, the present disclosure provides a wound dressing or wound healing agent comprises a peptide, wherein the peptide comprising: at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

According to an twelfth broad aspect, the present disclosure provides a method of wound treatment comprising: administering a hydrogel or organogel comprising a peptide to a subject via a device, wherein the peptide comprises: at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA, wherein X is comprised of a polar amino acid; and wherein n being an integer being selected from 0-5 and m being an integer being selected from 0-3.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

The accompanying drawings, which are incorporated herein and constitute part of this specification, illustrate exemplary embodiments of the invention, and, together with the general description given above and the detailed description given below, serve to explain the features of the invention.

FIGS. 1A through 1D illustrate ultrashort peptides self-assembled into three-dimensional nanofibrous networks (field emission scanning electron microscopy images of 4 mg/ml IVFK (FIG. 1A), 3 mg/ml IVZK (FIG. 1B), transmission electron microscopy images of silver nanoparticles formation in 4 mg/ml IVFK (FIG. 1C), and 3 mg/ml IVZK in water using mM AgNO₃ in Tris buffer at pH 8 (FIG. 1D)) according to one embodiment of the present invention.

FIGS. 2A through 2C illustrate a Rheology result of 5 mg/ml IVFK and IVZK peptide nanogels at a temperature of 40° C. ((FIG. 2A) Equilibrium moduli at 5 minutes, (FIG. 2B) oscillatory frequency sweep test, and (FIG. 2C) oscillatory amplitude sweep test) according to one embodiment of the present invention.

FIGS. 3A and 3B illustrate a graphical representation of an MTT assay using human dermal fibroblast cells in the presence of different peptide concentrations within a period of 24 hours (IVFK (FIG. 3A) and IVZK (FIG. 3B)) according to one embodiment of the present invention.

FIG. 4 illustrates a 3D viability assay of human dermal fibroblast cells encapsulated in peptide nanogels, 4 mg/ml IVFK and 3 mg/ml and 4 mg/ml Matrigel at different time points, such as at 3, 7, 14 and 21 days according to one embodiment of the present invention.

FIGS. 5A through SI illustrate cytotoxicity results of human dermal fibroblast cells encapsulated in peptide nanogels, 4 mg/ml IVFK and 3 mg/ml IVZK, and 4 mg/ml Matrigel, at different time points according to one embodiment of the present invention. Matrigel was used as a positive control (FIGS. 5A, 5D, 5G), IVFK (FIGS. 5B, 5E, 5H), and IVZK (FIGS. 5C, 5F, at days 7, 14 and 21, respectively. Scale bars 50 nm.

FIGS. 6A through 6I illustrate overlaid Z-stack confocal fluorescence images of human dermal fibroblast cells encapsulated in peptide nanogels (4 mg/ml IVFK and 3 mg/ml IVZK) and control Matrigel (4 mg/ml) at different time points according to one embodiment of the present invention. The Nucleus is shown in blue; F-actin is shown in red; and vinculin in green. Matrigel (FIGS. 6A, 6D, 6G), IVFK (FIGS. 6B, 6E, 6H), and IVZK (FIGS. 7C, 7F, 7I) nanogels on day 7, 14, and 21, respectively. Scale bar is 20 μm.

FIGS. 7A through 7D illustrate representative pictures of wound contraction after application of different peptide nanogels in comparison to standard care (FIG. 7A) according to one embodiment of the present invention. A quantitative evaluation of wound area contraction in response to different treatments (FIG. 7B) is illustrated according to one embodiment of the present invention. The percentage of re-epithelization (FIG. 7C) and granulation tissue formation reduction over the time (FIG. 7D) is shown, using digital planimetry according to one embodiment of the present invention. The error bars denote the standard error of the mean (n=10).

FIG. 8 illustrates a dropper/closure device for administering a hydrogel or organogel comprising the peptide according to one embodiment of the present invention according to one embodiment of the present invention.

FIG. 9 illustrates a squeeze bottle pump spray device for administering a hydrogel or organogel comprising the peptide according to one embodiment of the present invention according to one embodiment of the present invention.

FIG. 10 illustrates an airless and preservative-free spray device for administering a hydrogel or organogel comprising the peptide according to one embodiment of the present invention according to one embodiment of the present invention.

FIG. 11 illustrates an injectable device for administering a hydrogel or organogel comprising the peptide according to one embodiment of the present invention according to one embodiment of the present invention.

### DETAILED DESCRIPTION OF THE INVENTION

### Definitions

Where the definition of terms departs from the commonly used meaning of the term, applicant intends to utilize the definitions provided below, unless specifically indicated.

Unless defined otherwise, all technical and scientific terms used herein have the same meaning as is commonly understood to which the claimed subject matter belongs. In the event that there is a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

For purposes of the present invention, a value or property is “based” on a particular value, property, the satisfaction of a condition, or other factor, if that value is derived by performing a mathematical calculation or logical decision using that value, property or other factor.

For purposes of the present invention, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. For purposes of the present invention, directional terms such as “top,” “bottom,” “upper,” “lower,” “above,” “below,” “left,” “right,” “horizontal,” “vertical,” “up,” “down,” etc., are used merely for convenience in describing the various embodiments of the present invention. The embodiments of the present invention may be oriented in various ways. For example, the diagrams, apparatuses, etc., shown in the drawing figures may be flipped over, rotated by 90° in any direction, reversed, etc.

For purposes of the present invention, the term “comprising”, the term “having”, the term “including,” and variations of these words are intended to be open-ended and mean that there may be additional elements other than the listed elements.

For purposes of the present invention, the term “amino acid” refers to the molecules composed of terminal amine and carboxylic acid functional groups with a carbon atom between the terminal amine and carboxylic acid functional groups sometimes containing a side chain functional group attached to the carbon atom (e.g. a methoxy functional group, which forms the amino acid serine). Typically, amino acids are classified as natural and non-natural. Examples of natural amino acids include glycine, alanine, valine, leucine, isoleucine, proline, phenylananine, tyrosine, tryptophan, serine, threonine, cysteine, methionine, asparagine, glutamine, lysine, arginine, histidine, aspartate, and glutamate, among others. Examples of non-natural amino acids include L-3,4-dihydroxyphenylalanine, 2-aminobutyric acid, dehydralanine, g-carboxyglutamic acid, carnitine, gamma-aminobutyric acid, hydroxyproline, and selenomethionine, among others. In the context of this specification it should be appreciated that the amino acids may be the L-optical isomer or the D-optical isomer.

For purposes of the present invention, the term “biomolecule” refers to the conventional meaning of the term biomolecule, i.e, a molecule produced by or found in living cells, e.g., a protein, a carbohydrate, a lipid, a phospholipid, a nucleic acid, etc.

For purposes of the present invention, the term “carrier” refers to relatively nontoxic chemical compounds or agents that facilitate the incorporation of a drug into cells or tissues.

For purposes of the present invention, the term “cell-laden tissue scaffold” refers to the addition of cells on scaffold to form a tissue.

For purposes of the present invention, the term “effective amount” or “effective dose” or grammatical variations thereof refers to an amount of an agent sufficient to produce one or more desired effects. The effective amount may be determined by a person skilled in the art using the guidance provided herein.

For purposes of the present invention, the term “gel” and “hydrogel” are used interchangeably. These terms refer to a network of polymer chains, entrapping water or other aqueous solutions, such as physiological buffers, of over 99% by weight.

For purposes of the present invention, the term “microstructure” refers to a structure having at least one dimension smaller than 1 mm. A nanostructure is one type of microstructure.

For purposes of the present invention, the term “nanostructure” refers to a structure having at least one dimension on the nanoscale, i.e, a dimension between 0.1 and 100 nm.

For purposes of the present invention, the term “patient” and the term “subject” refer to an animal, which is the object of treatment, observation or experiment. By way of example only, a subject may be, but is not limited to, a mammal including, but not limited to, a human.

For purposes of the present invention, the term “pharmaceutically acceptable” refers to a compound or drug approved or approvable by a regulatory agency of a federal or a state government, listed or listable in the U.S. Pharmacopeia or in other generally recognized pharmacopeia for use in mammals, including humans.

For purposes of the present invention, the term “pharmaceutically acceptable carrier” refers to a carrier that comprises pharmceutically acceptable materials. Pharmaceutically acceptable carriers include but are not limited to saline solutions and buffered solutions. Pharmaceutically acceptable carriers are described for example in Gennaro, Alfonso, Ed., Remington's Pharmaceutical Sciences, 18th Edition 1990. Mack Publishing Co., Easton, Pa., a standard reference text in this field. Pharmaceutical carriers may be selected in accordance with the intended route of administration and the standard pharmaceutical practice.

For purposes of the present invention, the term “pharmaceutical composition” refers to a product comprising one or more active ingredients, and one or more other components such as carriers, stabilizers, diluents, dispersing agents, suspending agents, thickening agents, and/or excipients, etc. A pharmaceutical composition includes enough of the active object compound to produce the desired effect upon the progress or condition of diseases and facilitates the administration of the active ingredients to an organism. Multiple techniques of administering the active ingredients exist in the art including, but not limited to: topical, ophthalmic, intraocular, periocular, intravenous, oral, aerosol, parenteral, and administration. By “pharmaceutically acceptable,” it means the carrier, diluent or excipient must be compatible with the other ingredients of the formulation and not deleterious to the recipient thereof, i.e, the subject.

For purposes of the present invention, the term “room temperature” refers to a temperature of from about 20° C. to about 25° C.

For purposes of the present invention, the term “scaffolds” as used herein means the ultra-short peptide or other polymer materials in the bioinks that provide support for the cellular components.

For purposes of the present invention, the term “seeding” refers to a method to add cells on a scaffold to produce surfaces.

For purposes of the present invention, the term “subject” and the term “patient” refers to an entity which is the object of treatment, observation, or experiment. By way of example only, a “subject” or “patient” may be, but is not limited to: a human, a mammal, a reptile, a bird, a fish, an amphibian, and an invertebrate.

For purposes of the present invention, the term “therapeutically effective amount” and the term “treatment-effective amount” refers to the amount of a drug, compound or composition that, when administered to a subject for treating a disease or disorder, or at least one of the clinical symptoms of a disease or disorder, is sufficient to affect such treatment of the disease, disorder, or symptom. A “therapeutically effective amount” may vary depending, for example, on the compound, the disease, disorder, and/or symptoms of the disease or disorder, severity of the disease, disorder, and/or symptoms of the disease or disorder, the age, weight, and/or health of the subject to be treated, and the judgment of the prescribing physician. An appropriate amount in any given instance may be readily ascertained by those skilled in the art or capable of determination by routine experimentation.

For purposes of the present invention, the term “ultra-short peptide” and “self-assembling peptide” are used interchangeably. These terms refer to a sequence containing 3-7 amino acids.

### DESCRIPTION

While the invention is susceptible to various modifications and alternative forms, specific embodiments thereof has been shown by way of example in the drawings and will be described in detail below. It should be understood, however that it is not intended to limit the invention to the particular forms disclosed, but on the contrary, the invention is to cover all modifications, equivalents, and alternatives falling within the spirit and the scope of the invention.

Non-healing chronic wounds are severe complications, which can often eventually lead to amputations. As such, there is a clear clinical need for dressings that promote the healing of chronic wounds. An advanced wound dressing aims to keep wound tissues moist while offering increased healing rates, preventing scar formation, reducing pain, minimizing infection, improving cosmetics, and lowering overall health care costs. Disclosed embodiments have previously developed tetrameric peptides Ac-IVZK-NH₂ (Ac-Ile-Val-Cha-Lys-NH₂) and Ac-IVFK-NH₂ (Ac-Ile-Val-Phe-Lys-NH₂) that self-assemble into nanofibrous hydrogels with biomimetic properties resembling those of collagen. In the disclosed study, embodiments tested if these nanogels can fulfill the wound healing criteria mentioned above and found that the nanogels are suitable scaffolds for encapsulating human dermal fibroblasts. Disclosed embodiments selected peptide nanogels Ac-IVZK-NH₂ and Ac-IVFK-NH₂ and produced silver nanoparticles in situ within the nanogels to assess their efficacy on micropigs with full-thickness excision wounds. The in-situ generation of the silver nanoparticles was done solely through UV irradiation; no reducing agent was used. Application of the peptide nanogels on full thickness micropig wounds demonstrated that the scaffolds are biocompatible and did not trigger wound inflammation. This suggests that the scaffolds are safe for topical application. A comparison of the effect of both nanogels even without the addition of the silver nanoparticles, revealed that the scaffold itself has a high potential to act as an antibacterial agent, which may suppress both the inflammatory reaction and the activity of proteases. Interestingly, the effect on wound closure of the peptide nanogels was comparable to those of standard care hydrogels. Despite the disclosed promising results, there is still much to learn about the molecular basis underlying the efficacy of tetrameric peptide nanogels in wound healing. This will support the urgent demand for advanced treatments of diabetic wounds, based on scientifically and clinically validated studies.

An injury affecting the skin's protective barrier and function can be defined as a wound [1]. Skin injuries are further classified into two categories based on the nature of the restoration process. Acute wounds typically arise from tissue injuries after frictional contact between the skin and hard surfaces and heal with minimal scarring within a short period of time [2]. Chronic wounds, on the other hand, result from repeated tissue injuries or underlying physiological conditions, taking at least 12 weeks for full healing such as in chronic diabetic ulcers and malignancies [3]. Wound healing is a complex energetic process consisting of four main steps: coagulation and hemostasis, inflammation, proliferation (granulation and contraction), and finally remodeling. In chronic wounds, the wound remains in the proliferation stage with excess inflammation and a failure to progress to remodeling [4]. The challenge of delayed wound healing has been the focus of many research studies. Existing literature provides a broad spectrum of treatment modalities striving to improve the healing process in chronic wounds. One method is the application of tissue engineering principles to facilitate the re-establishment of tissue integrity [5]. Bell and his group were the first to use composites with both dermal and epidermal components for treating patients with chronic burn wounds, but the results were not satisfactory [6]. They have also developed a matrix containing fibroblast/collagen known as Apligraft® [7].

Various dressings have been developed to avoid infection and enhance wound healing [8-10], such as collagen/polycaprolactone polymers and sponges with bovine collagen I/chondroitin-4-6 and sulfate/chitosan [12]. Peptide nanogels offer a novel approach to enhance wound healing by improving existing synthetic materials, the majority of which have been polymers [1]. Peptide can have intrinsic antimicrobial and anti-inflammatory activity, which are crucial as the prolonged existence of infectious pathogens can result in a delayed healing process [13,14]. Peptides are biocompatible and offer tunable biodegradability [13,14]. Also, peptide nanogels combine the benefits of commercially available hydrogel dressings with those of nanofibrous scaffolds in providing the structural cues necessary for tissue restoration, in addition to providing an optical transparency that enables the observation of the wound. The soft properties of peptide nanogels reduce the pain that regularly occurs during routine wound dressing changes [1]. Different studies have investigated the use of hydrogel scaffolds as wound dressings and carriers of components that can promote healing. Studies on their effect on chronic wounds are scarce, however, and not satisfactory. The ideal dressing for chronic wounds should be free from contaminants and eliminate excess wound exudates, among other toxic components. Also, the dressing should maintain a moist environment, prevent the entrance of microorganisms, allow gaseous exchange, facilitate autolytic debridement, enhance granulation tissue formation, and provide cues to improve tissue regeneration [10]. In the past, nanofibrous silk materials and different electrospun polymers have been used as wound dressings, but, unlike hydrogel dressings, they failed to provide the moist environment necessary for cellular proliferation and debridement. Furthermore, natural and synthetic hydrogels made from alginate, chitosan, hyaluronic acid, and polyethylene glycol, do not produce nanofiber networks similar to the fibers found in the extracellular matrix (ECM) during their self-assembly processes, rather the resulting fibers are significantly larger, with dimensions on the microscale [17].

Disclosed embodiments focused on the development of novel synthetic peptide-based biomaterials [18,19] that aim to combine the advantages of both natural and synthetic hydrogels. Disclosed embodiments have rationally designed and synthesized various different peptides with sequences consisting of three to seven amino acid residues. These peptides can self-assemble in water to form transparent nanofibrous hydrogels [20,21]. Among them, some amphiphilic ultrashort peptides have been shown to accelerate skin regeneration when used as dressings for burn wounds [1].

In this study, disclosed embodiments have used previously designed tetrameric peptides that self-assemble into nanogels to evaluate their efficacy in treating full thickness wounds in micropigs. The nanofibrous network structure was confirmed by scanning electron microscopy (SEM). Furthermore, in situ silver nanoparticle formation within the transparent hydrogels were examined by transmission electron microscopy (TEM). The biocompatibility of these nanofibrous hydrogels was tested on human dermal fibroblast (HDFn) to further assess the candidacy of these novel biomaterials for wound healing applications. Cell distribution, alignment, and phenotype were studied within a 3D culture environment by staining the actin cytoskeleton. Disclosed embodiments have found that both peptide nanogels supported the proliferation of HDFn for up to three weeks and were thus deemed safe for topical application. To study the peptide nanogels in vivo, disclosed embodiments topically applied them and their silver nanoparticle encapsulated counterparts to full thickness incision wounds in micropigs. Disclosed embodiments examined their healing capacity in comparison to commercially available standard of care hydrogel dressings, namely DuoDerm® Hydroactive® hydrogel and AQUACEL® Ag EXTRA™-Hydrofiber™. The results showed a comparable effect when using these biomaterials both with and without silver nanoparticles (AgNPs) as well as the equivalent controls, thus demonstrating the biocompatibility of these materials and their ability to act as antibacterial agents. Disclosed embodiments propose that the peptide nanofibers were integrated into the wound area, thereby merging with existing ECM fibers and providing additional structural and functional support.

Impairment of wound healing in patients with complicated chronic wounds as well as in patients with diabetic ulcers affects the quality of life of millions of people and requires a high cost to cure. Wound healing is a complicated process that relies on communication between different cell types, growth factors, and extracellular matrix molecules. Various dressings have been developed to enhance wound healing in individuals without co-morbidities, typically based on enhancing debridement, cleaning and offering a moist environment conducive to healing.

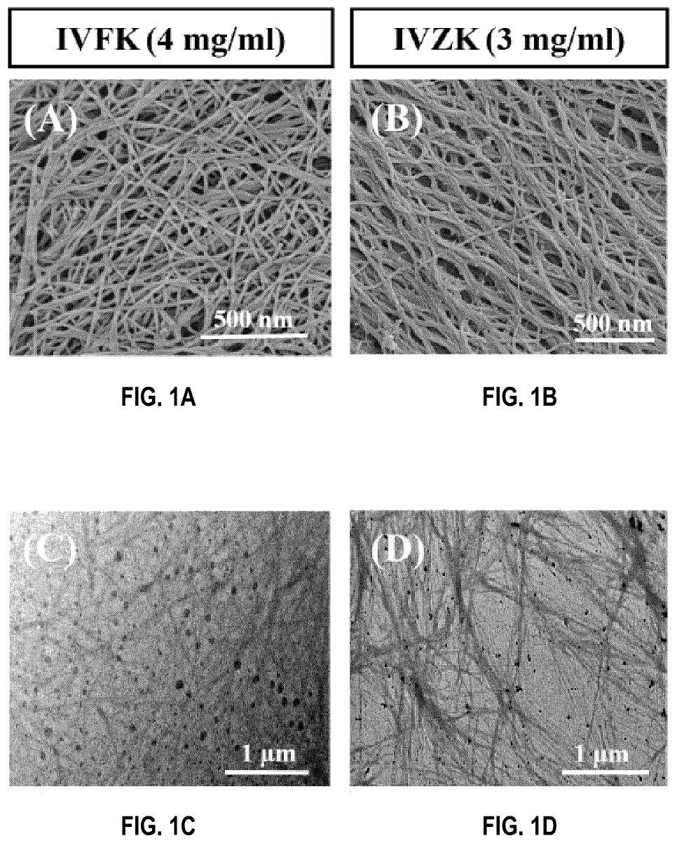

Herein, disclosed embodiments evaluated the efficiency of previously designed tetrameic ultrashort aliphatic peptide hydrogels (IVFK and IVZK) as primary dressings for full thickness wounds. These peptides have a native ability to self-assemble into fibrous scaffolds in aqueous solution. SEM confirmed a dense nanofibrous network formation (FIGS. 1A and 1B) where the fibers structurally resemble collagen fibers with respect to topography [25]. This suggests their use as scaffolds for skin regeneration [26-29], particularly for full thickness wounds. The diameter of these nano-scale fibers ranges within the diametric scope found in the natural ECM (5-300 nm) [30]. This distinctive fibrous structure allows the gel to entrap >99% water within its bulk volume. It preserves a moist environment and leaves the tissue hydrated which in turn reduces the pain during frequent dressing changes [1]. Additional advantages of the peptide nanogels are their optical transparency that allows for observation of the wound. Another advantage of selecting these peptides is that the self-assembly can be enhanced in the presence of phosphate buffer saline (PBS), which usually is used to prepare wound dressings. This preserves the physiological pH and enhances the wound dressing capacity to absorb wound exudates. Importantly, the short length of the peptides offers the advantage of a low cost production different to other protein- or polypeptide-based hydrogels. Also, these nanogels comprise a polar lysine amino acid residue, which aids the wound homeostasis [31]. In addition, the porous nanofibrous structure supports the in situ fabrication of silver nanoparticles (AgNPs) from a silver nitrate solution without using any chemical reducing agent (FIG. 1C and FIG. 1D). TEM confirmed monodispersed AgNPs with a size range of approximately 10-20 nm, which has been verified as a sufficient size to elicit antibacterial activity [32].

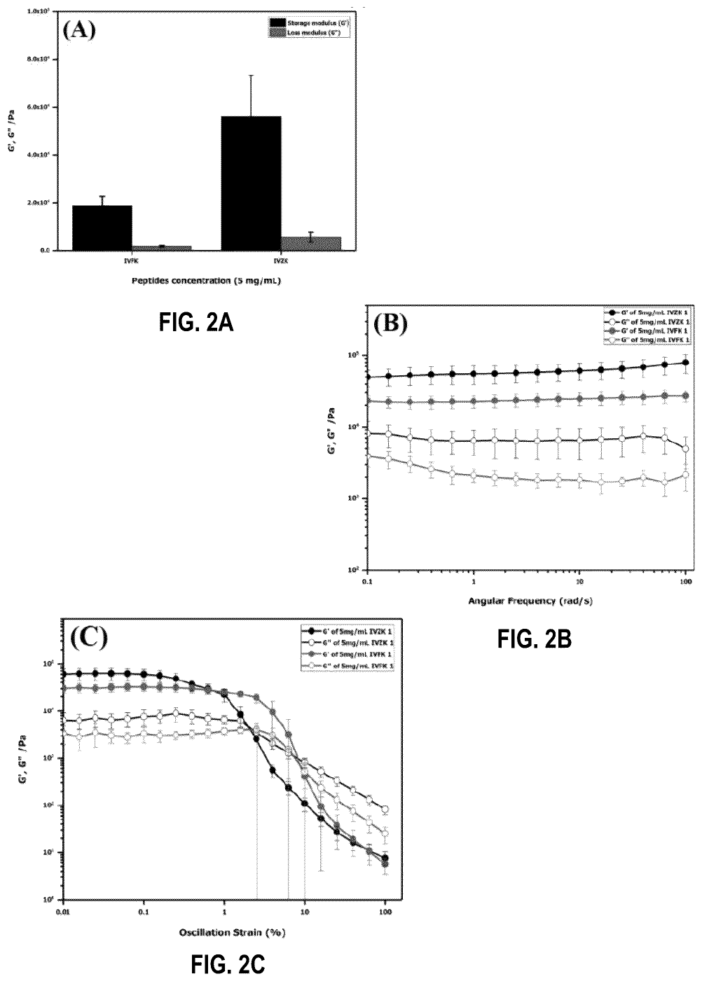

The mechanical stiffness and stability of both peptide nanogels were determined using oscillatory rheology that is based on measuring the storage modulus (G′) and loss modulus (G″). Samples were examined through time-sweep analysis in a linear viscoelastic range (LVR) for five minutes by keeping the storage modulus constant under elastic deformation. The G′ values of IVFK and IVZK were found to be around 18.9 kPa and 56.1 kPa, respectively, while their G″ values were less than an order of magnitude from their G′ indicating the gel state of both samples, as shown in FIG. 2A [33]. Both samples also showed a frequency-independent behavior, without any crossover at lower frequencies, which is a common viscoelastic property for hydrogel (FIG. 2B) [34]. An amplitude sweep test demonstrated the length of the LVR under increasing strain values. It was found that the LVR for both peptide gels was still constant under 0.2% strain. However, the later cross-over point of IVFK where G′=G″ indicated that IVFK could tolerate higher strain before the breaking of the gels, in addition to its high G′ value (FIG. 2C) [35].

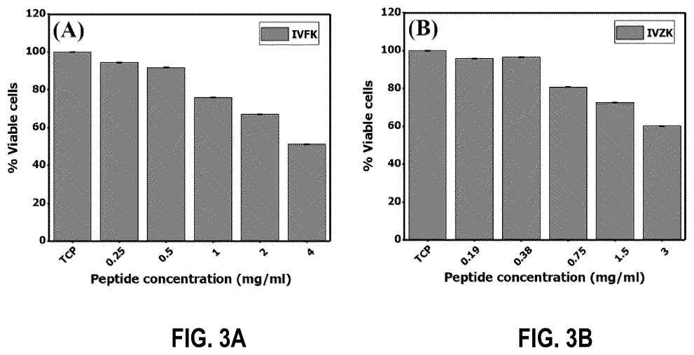

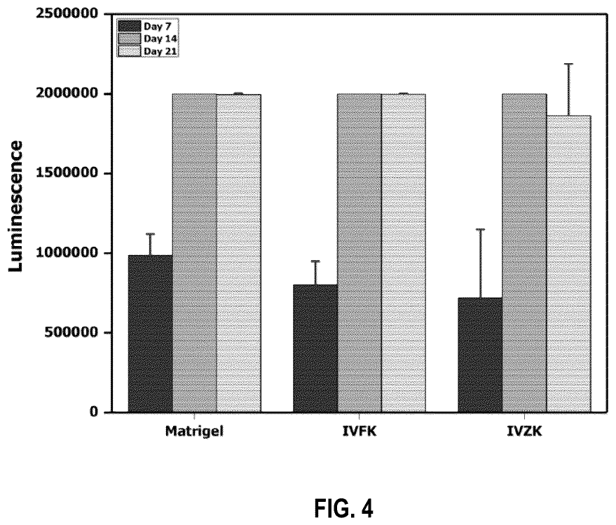

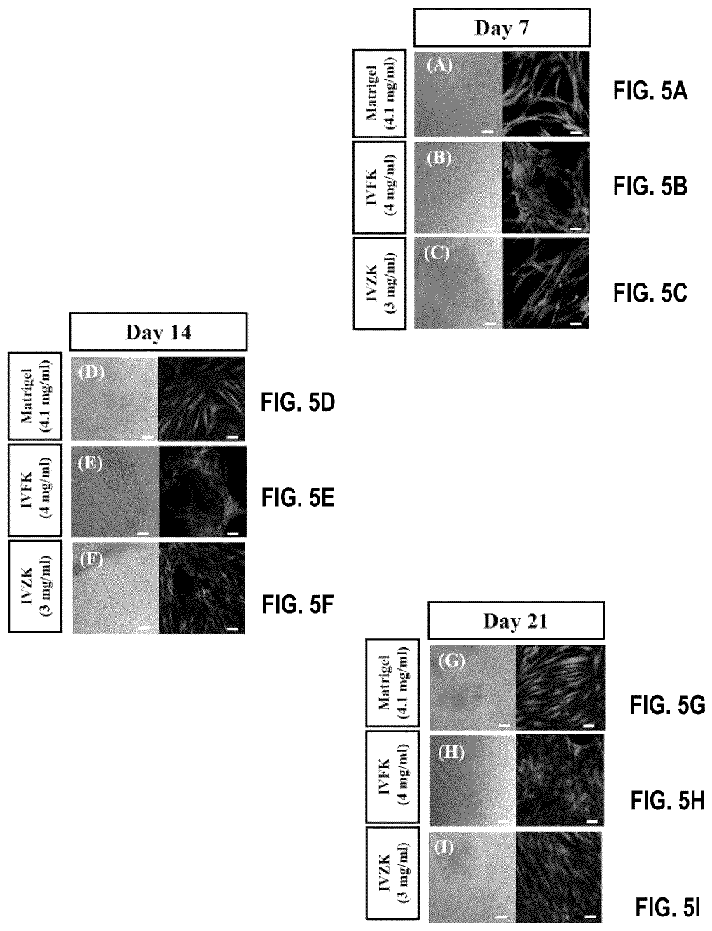

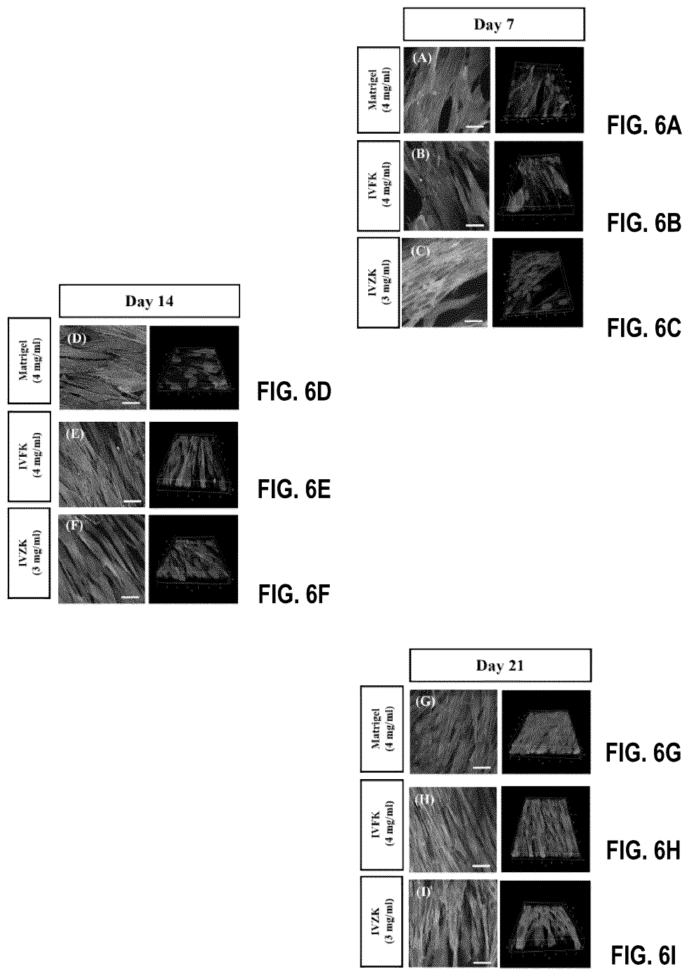

As cellular proliferation, adhesion and the formation of three-dimensional cellular networks are extremely important for tissue repair and regeneration, cytocompatibility of the peptide nanogels were evaluated using neonatal human dermal fibroblast cells. The in vitro investigation demonstrated that exposure of HDFn to different concentrations of peptide nanogels did not affect cell growth when compared to cell growth in tissue culture plates (TCP), as shown in FIG. 3. Further, a time-dependent increase in ATP production was observed in 3D cultured HDFn for 7, 14, and 21 days (FIG. 4), demonstrating that the encapsulated human dermal fibroblasts were metabolically active up to three weeks. Moreover, the fluorescence intensity of green color resulting from the cytotoxicity assay revealed that the majority of cells were alive up to 21 days, with very few dead cells (FIG. 5). Furthermore, fluorescence confocal z-stack images of the actin cytoskeleton, which gives direct evidence for cellular morphology and cytoskeleton structure [36], demonstrated that after a period of seven days, cells proliferated and extended within the scaffolds (FIGS. 6A-6C). At day 14, the fibroblast growth rapidly increased and created a network via cell-to-cell junctions (FIGS. 6D-6F). Continuous growth of encapsulated cells and the formation of an extensive network saturating the nanogel matrix, was observed at day 21 (FIGS. 6G-6I). Upon this observation, in accordance with disclosed embodiments, the peptide scaffolds provided a favorable microenvironment for cell adhesion, spreading and proliferation. Therefore, it is hypothesized that compatible peptide biomaterials may have an ability to boost the healing of chronic wounds and enhance tissue regeneration.

Disclosed embodiments provided an in vivo wound healing study, wherein micropigs were used as a suitable animal model, because pigs, although a costly animal model, resemble humans regarding skin structure and wound healing characteristics [37]. Sullivan, et al. have considered pigs as a satisfying animal model for research related to skin, when comparing the outcomes obtained on clinical studies, and the results obtained on pigs, small mammals, as well as within in vitro studies [38]. Further, the pre-clinical porcine model has efficiently been used to assess the safety and efficacy of peptide nanogels for wound healing, due to its anatomical and functional similarity to humans when compared to other animal models [39]. Applying 25 different wound therapies using porcine models and comparing it with studies on humans revealed that the data of 78% of the porcine studies agreed with those of the human studies, whereas only 53-57% showed agreement when using small mammal models [38].

Full-thickness incision wounds (1×1 cm) were created on the dorsal back of all micropigs by surgical removal of the upper epidermis layer and middle dermis layer. The newly created wounds were used to evaluate the therapeutic effects of different biomaterials on the wound closure. A block of different hydrogel dressing (IVFK, IVZK, IVFK-AgNPs, IVZK-AgNPs) or DuoDerm® and Aquacel® (positive controls) was topically applied. Then, Tegaderm was used to protect and cover wounds and to allow gaseous exchange.

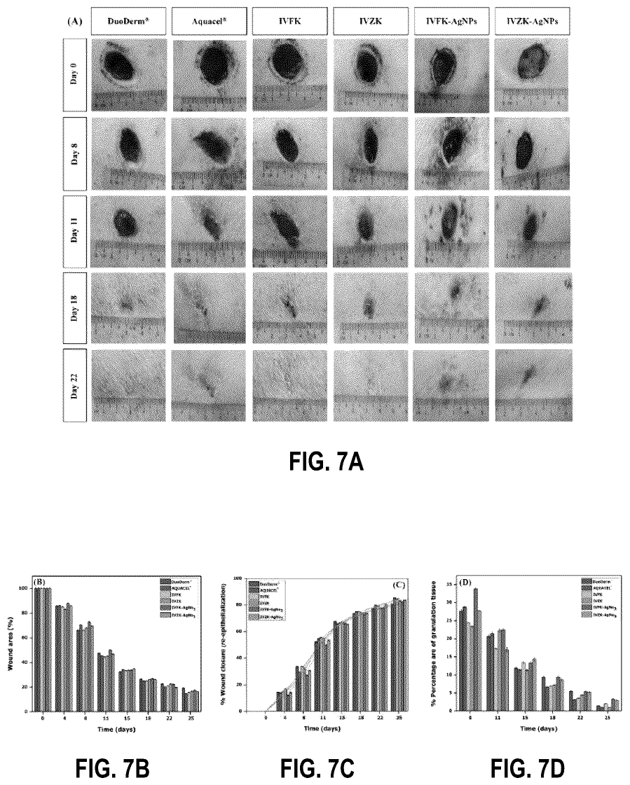

After each wound dressing change, digital planimetry was applied to quantify the re-epithelialization and granulation tissue formation. Re-epithelialization is very important for protecting the host against pathogens as the prolonged exposure to pathogens can cause excessive inflammation that affects the organ functions and delays or undermines the skin regeneration process [1]. Granulation tissue, on the other hand, known to occur with proliferating fibroblast and formation of newly microvasculature was observed in some pigs in their open wounds at day 8, with a percentage of 26.7%, 28.8%, 24.5%, 23.4% 33.8% and 27.8% for duoderm, aquacel, IVFK, IVZK, IVFK-AgNPs and IVZK-AgNPs, respectively (FIG. 7D). The granulation tissue formation was denser in the IVFK-AgNO₃ treated group compared to the controls and other treated hydrogels groups. However, the difference did not reach statistical significance. After 11 days of treatment, enhanced re-epithelialization and formation of granulation tissue at the wound edge, when compared to the center of the wound, was observed in all studied micropigs, as shown in FIGS. 7C and D. Nearly complete re-epithelialization was achieved at day 22 with a gradual reduction in granulation tissue formation. The presence of granulation tissue suggests that both hydrogels support the proliferation of endothelial cells and thus enhance angiogenesis.

Statistical analysis revealed that after four days of treatment the percentage of the wound area was slightly reduced by IVFK hydrogels (79.2%) and IVZK hydrogels (79%), compared with duoderm (83%), aquacel (81%), IVFK-AgNPs (91.7%) and IVZK-AgNPs (81.4%). However, the reduction was not significant (FIGS. 7A, B). Chronic wounds are characterized by a prolonged inflammatory reaction, which results in imbalances in macrophage functions leading to a significant decrease in the pro-healing macrophage phenotype, when compared to the pro-inflammatory macrophage which results in increased protease secretion at the wound site [41,42]. Silver-based antibiotics were used to prevent the infection of the wound [43,44]. It is worth mentioning that both peptide nanogels with and without silver nanoparticles had a similar effect on wound closure, demonstrating that these hydrogels have intrinsic antimicrobial activity comparable to the silver nanoparticles that suppress the inflammatory response and inhibit the activity of the protease. Also, there were no visually apparent differences among all tested conditions, including both controls, peptide nanogels with and without silver nanoparticles in respect to minimal scar formation and negligible infection.

The application of these hydrogels to chronic full-thickness wounds stimulate granulation tissue formation as well as the re-epithelialization which eventually closes the wound without the addition of exogenous growth factors or cells [45]. Also, the observed ability of these peptide nanogels alone, without any additional additive factors, to exhibit a similar effect as the controls will enable future development of 3D scaffolds containing skin/stem cell as well as angiogenic factors to promote vascularization that is necessary for successful tissue engineering graft. Moreover, these peptide nanogels, containing only four amino acids, are less costly and devoid of any chemical additive, in contrast to commercially available hydrogel dressings. Considering the results of the disclosed embodiments, it is believed that the utilized peptide nanogels are promising materials for fabricating skin substitutes as well as 3D skin graft models, particularly in the context of wound healing.

This disclosed study is giving preliminary in vitro and in vivo evidence on the efficacy of the investigated peptide nanogels as promising wound dressings. Further studies are necessary and mandatory to check closely and in more detail on wound hydration, autolytic debridement, inflammation, cytokine expression, and metalloproteinases activity.

The disclosed study shows that newly developed peptide nanogels provide native cues to human dermal fibroblast cells and promote their proliferation as well as their extensive network formation in vitro. The application of the disclosed ultrashort peptide nanogels on full thickness wounds in a minipig model demonstrated biocompatibility with micropig skin tissue. The peptide nanogels did not trigger wound inflammation and thus can be considered to be safe for topical applications. The peptide-based biomaterials exhibited a similar effect in wound closure when compared to the current standard of care hydrogels. Moreover, the comparable effect obtained after using hydrogels with/without AgNPs indicated that these materials have a high potential to act as an antibacterial agent. The disclosed results suggest that peptide nanogels as a wound dressing have the prospect to strongly enhance chronic wound healing when combining with cells or growth factors that are suitable for skin tissue regeneration. Disclosed embodiments propose that the peptide nanogels could act as potential carriers for HDFn transplantation in in vivo therapies and as promising biomaterials for tissue engineering applications. Further in vivo studies should be performed to assess how the 3D culture peptide scaffolds work when seeded together with autologous skin cells. Follow-up studies are critically needed as they will allow for a more precise evaluation of the dressings' fate post-grafting.

In one embodiment, a peptide capable of forming a gel by self-assembly comprising at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

In one embodiment, amino acids in the peptide are either L-amino acids or D-amino acids.

In one embodiment, a hydrogel or organogel comprises a peptide, wherein the peptide comprises at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

In one embodiment, a hydrogel or organogel is comprised in at least one of a fuel cell, a solar cell, an electronic cell, a biosensing device, a medical device, an implant, a pharmaceutical composition and a cosmetic composition.

In one embodiment, N-terminus of the peptide is modified with a functional group selected from the group consisting of carboxylic acid, amide, alcohol, aldehyde, amine, imine, nitrile, an urea analog, phosphate, carbonate, sulfate, nitrate, maleimide, vinyl sulfone, azide, alkyne, alkene, carbohydrate, imide, peroxide, ester, aryl, ketone, sulphite, nitrite, phosphonate, and silane.

In one embodiment, C-terminus of the peptide is functionalized, by chemical conjugation or coupling of at least one compound selected from bioactive molecules or moieties, wherein the bioactive molecules or moieties are selected from the group consisting of growth factors, cytokines, lipids, cell receptor ligands, hormones, prodrugs, drugs, vitamins, antigens, antibodies, antibody fragments, oligonucleotides, and saccharides, and wherein the chemical conjugation can be carried out before or after self-assembly wherein the chemical conjugation can be carried out before or after self-assembly.

In one embodiment, a peptide is employed in at least one of the group consisting of a medical tool kit, a fuel cell, a solar cell, an electronic cell, regenerative medicine and tissue regeneration, implantable scaffold disease model wound healing, 2D and 3D synthetic cell culture substrate, stem cell therapy, injectable therapies, biosensor development, high-throughput screening, biofunctionalized surfaces, printing biofabrication, bio-printing, and gene therapy.

In one embodiment, a method of preparing a hydrogel or organogel, the method comprising dissolving a peptide in an aqueous solution or an organic solution, respectively, wherein the peptide comprises: at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

In one embodiment, a cell or tissue graft comprising a hydrogel, wherein the hydrogel comprises a peptide capable of forming a gel by self-assembly comprising: at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

In one embodiment, a wound dressing comprising a hydrogel, wherein the hydrogel comprises at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

In one embodiment, a surgical implant comprising a peptide scaffold, wherein the peptide scaffold is formed by a peptide capable of forming a gel by self-assembly comprising at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

In one embodiment, a pharmaceutical composition for treating a wound comprises a peptide capable of forming a gel by self-assembly comprising at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA, wherein X is comprised of a polar amino acid; and wherein n being an integer being selected from 0-5 and m being an integer being selected from 0-3.

In one embodiment, the pharmaceutical composition, further comprising a pharmaceutically active compound.

In one embodiment, the pharmaceutical composition is provided in a form of at least one selected from the group consisting of a topical gel, a topical cream, a spray, a powder, a sheet, a patch, a membrane, and an injectable solution.

In one embodiment, the pharmaceutical composition, further comprising a pharmaceutically acceptable carrier.

In one embodiment, the peptide is at least one selected from the group consisting of IVFK and IVZK.

In one embodiment, a kit comprising a first container with a peptide and a second container with an aqueous or organic solution, wherein the peptide comprises at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

In one embodiment, the kit, wherein the aqueous or organic solution of the second container further comprises a pharmaceutically active compound, and wherein the first container with a peptide further comprises a pharmaceutically active compound.

In one embodiment, a bioimaging device comprises a peptide capable of forming a gel by self-assembly comprising at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

In one embodiment, a 2D or 3D cell culture substrate comprises a peptide capable of forming a gel by self-assembly comprising at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA, wherein X is comprised of a polar amino acid; and wherein n being an integer being selected from 0-5 and m being an integer being selected from 0-3.

In one embodiment, a wound dressing or wound healing agent comprises a peptide, wherein the peptide comprising: at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

In one embodiment, a method of wound treatment comprising: administering a hydrogel or organogel comprising a peptide to a subject via a device, wherein the peptide comprises: at least one peptide selected from a group of peptides having a formula selected from AₙBₘX, BₘAₙX, XAₙBₘ, and XBₘAₙ; wherein A is an aliphatic amino acids; wherein B is comprised of at least one aromatic amino acid selected from the group consisting of: tyrosine, tryptophan, phenylalanine, and L-DOPA; wherein X is comprised of a polar amino acid; and wherein n being an integer being selected from 0-5 and m being an integer being selected from 0-3.

In one embodiment, a device is selected from the group consisting of a container with a dropper/closure device, a squeeze bottle pump spray, an airless and preservative-free spray, and an injectable device.

In one embodiment, the subject is a human.

In one embodiment, the subject is selected from the group consisting of a mammal, a reptile, a bird, a fish, an amphibian, and an invertebrate.

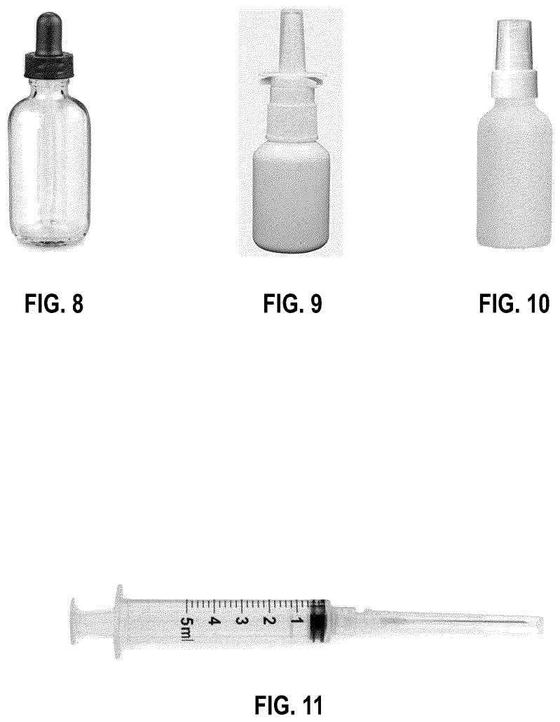

While preferred methods and devices of the present invention may include the device selected from the group consisting of a container with a dropper/closure device (FIG. 8), a squeeze bottle pump spray (FIG. 9), an airless and preservative-free spray (FIG. 10), and an injectable device (FIG. 11), it is readily appreciated that skilled artisans may employ other means and techniques for delivering the peptide-based adhesive material.

The injectable device (FIG. 11) may not be limited to syringe-type device. One of ordinary skill in the art would readily appreciate that any injectable device suitable for delivering the peptide-based adhesive material may be utilized according to aspects of the present invention.

One of ordinary skill in the art would readily appreciate that any kind of device suitable for delivering the disclosed products described in the present invention may be utilized.

Having described the many embodiments of the present invention in detail, it will be apparent that modifications and variations are possible without departing from the scope of the invention defined in the appended claims. Furthermore, it should be appreciated that all examples in the present invention, while illustrating many embodiments of the invention, are provided as non-limiting examples and are, therefore, not to be taken as limiting the various aspects so illustrated.

### EXAMPLES

### Materials

Two tetrameric self-assembling peptides IVFK (Ac-Ile-Val-Phe-Lys-NH₂) and IVZK (Ac-Ile-Val-Cha-Lys-NH₂) were custom synthesized by Bachem AG, (Budendorf, Switzerland). Neonatal human dermal fibroblast cells (HDFn) were purchased from Gibco (Grand Island, USA). Dulbecco's Modified Eagle Medium (DMEM), fetal bovine serum (FBS), Dulbecco's phosphate-buffered saline (PBS) solution, and penicillin-streptomycin antibiotics (P/S) were purchased from Gibco (Grand Island, USA). CellTiter-Glo® Luminescent 3D cell viability assay kits were ordered from Promega (Madison, USA). MTT Cell Proliferation Assay kit and LIVE/DEAD® Viability/Cytotoxicity kit was purchased from either ThermoFisher Scientific, USA, Promega, USA or Life Technologies™, USA. Immunostaining antibodies antivinculin and Rhodamine-Phalloidin were purchased from Invitrogen, USA, and anti-mouse IgG-FITC was purchased from Sigma, USA. T175 or T75 cell culture flasks and 96-well plates were ordered from Corning, USA.

### Methods

### Peptides Hydrogel Preparation

IVZK and IVFK peptide powders were dissolved in Milli-Q water. Then, 10× phosphate buffered saline was added to the peptide solution. Gelation of both peptides occurred within a few seconds at minimum concentration of 4 mg/mL and 3 mg/mL for IVFK and IVZK, respectively. The final volume ratio of peptide solution and 10×PBS was 9:1.

### Evaluation of Fiber Structures by Field-Emission Scanning Microscopy (FE-SEM)

The peptide nanogels were dehydrated by gradually increasing concentrations of 30%, 50%, 70%, 90% and 100% (v/v) ethanol solutions for 15 mins in each solution. Further dehydration in 100% ethanol solution was continued by changing the absolute ethanol solution with a fresh one twice for 15 mins each. The dehydrated samples were subsequently kept in 1:2 (v/v) hexamethyldisilazane (HMDS): ethanol for 20 min, followed by 20 mins of incubation in a fresh solution of 2:1 HMDS: ethanol and then two times of 20 mins in 100% HMDS. Finally, the samples were kept inside a fume hood overnight to allow HMDS to evaporate. Prior to imaging, the samples were mounted onto SEM stubs using conductive carbon tape, and then sputter-coated with a 5 nm thick coating of Iridium and a 3 nm thick coating of Gold/Palladium. The coated samples were then imaged with a Field Emission Scanning Electron Microscopy system (FEI Nova Nano630 SEM, Oregon, USA).

### Evaluation of Silver Nanoparticles (AgNPs) Formation by Transmission Electron Microscopy (TEM)

Silver nanoparticle studies were carried out as previously described [23]. Briefly, the peptide powders were dissolved in distilled water. Then, 100 mM of silver nitrate (AgNO3) in Tris buffer (pH=8.5) was added into the peptide solution for a final concentration of 10 mM. Gelation occurred within 5 minutes at the minimum concentration of 4 mg/mL IVFK and 3 mg/mL IVZK. The gels were then exposed to 254 nm UV light for 5 minutes allowing for in situ formation of AgNPs. A small portion of the gel was taken and diluted in water. Then, 10 ul of this diluted gel was dropped onto carbon-coated cupper grid and kept there for 5 min before blotting with filter paper. The grid was then dried overnight at RT. TEM analysis was performed with the Tecnai G2 Spirit Twin instrument with an accelerating voltage of 120 kV using an emission gun.

### Rheology Studies

The viscoelastic properties of peptide nanogels were characterized by using the TA Ares-G2 rheometer with an 8 mm parallel-plate geometry. The peptide gels were prepared in a 8 mm diameter polymethylmethacrylate ring, which were later being incubated inside a sealed tissue-culture dish at 40° C. overnight before the measurements. For each peptide, six replicates of gels having a volume of 150 μL were prepared. The measurements were conducted at a 1.5 mm gap distance with a temperature of 40° C. to mimic conditions used for cell growth. Three consecutive tests, such as time-sweep, frequency-sweep, and amplitude-sweep, were carried out for each sample to confirm the mechanical stiffness of the gels. Time-sweep analysis was performed for 5 minutes at an angular frequency of 1 Hz and at 0.1% strain to observe storage and loss modulus at an early stage, before the next measurements were done. A frequency sweep analysis was subsequently carried out by reducing the angular frequency from 100 to 0.1 Hz while keeping the strain at 0.1%. Lastly, amplitude sweep analysis was performed to observe the minimum value of the amplitude strain needed to break the gel structure. This test was performed with an angular frequency and strain of 1 Hz and 0.01%-100%, respectively.

### Cell Culture

Human dermal fibroblast (HDFn) was cultured either in T175 or T75 culture flasks in complete DMEM media (10% fetal bovine serum, and 1% penicillin/streptomycin). The cells were incubated in a humidified incubator with 95% air and 5% CO2 at 37° C. The cells were sub-cultured using trypsin at approximately 80% confluence. The culture media was replenished every 48 h.

### Establishing 3D Culture

3D cell culture was performed as previously described [22,24]. Briefly, a gel base at the bottom of a cell culture plate was created by pipetting a peptide solution (peptide with water) into a glass bottom dish (Nunc, 12 mm) and mixing it with 2×PBS. After gel formation, the peptide solution was pipetted on top of this gel base and mixed with human dermal fibroblasts (25,000 cells/dish) suspended in 2×PBS. Gelation occurred within 3-5 minutes. Then, the culture medium was subsequently added on top of each construct. The plate was incubated at 37° C., 95% air and 5% CO2. Different biocompatibility assays were applied using this construct.

### Biocompatibility Evaluations of Peptide Nanogels

All the biocompatibility studies, including cell viability assay (MTT assay), ATP production (3D cell proliferation assay), and live/dead assay, as well as morphological changes in response to peptide nanogels, were performed as previously described [22,24].

### In Vivo Wound Healing Evaluation by Peptide Nanogels in Micropigs

The study was designed as a case-control study. All experimental protocols and animal care complied with the “Guide for the care and use of Laboratory Animals” (PWG, 15 Tech Park Cres, Singapore 638117; Prof Lim Thiam Chye, Department of Surgery, Yong Loo Lin Medical School, National University of Singapore).

### Animals and Maintenance

A total number of 10 healthy micropigs (Sus Scrofa), (5 male and 5 female PWG micropigs) of comparable age (adult, over 16 months), with a weight of approximately 25-30 kg at the start of the experiment, were selected for the wound healing study. Before initiating the experiment, the animals were acclimatized for 3-7 days. During this period, all animals were observed daily for any clinical signs of disease. A unique number associated with the ear-tag number identified each animal. Then, all pens were labeled with a cage card indicating study number, and animal ID. The cages are equipped with food bowls and water bottles. The room temperature was 22-30° C. with 50-80% humidity and approximately 12 hours of 150-300 lux of the light/dark cycle. Each animal was fasted overnight (at least 12 hours) before the experimental procedures and deprived of food during wound creation and dressing. After wound creation, each animal was individually housed, using steel cages with a size of approximately 80 cm×140 cm×75 cm and defined by a marked cage card.

### Wounding and Chamber Treatment

For all surgical procedures, intramuscular Ketamine eats 10 mg/kg, Atropine at 0.05-0.10 mg/kg and Xylazine eats 2-5 mg/kg intra-muscularly was administered at the time of anesthetic induction. General anesthesia was maintained with 2-5% isoflurane in oxygen. The anesthetized pigs were placed in stern recumbency, and the hair removed, as required. Care was taken to avoid mechanical irritation and trauma. The surgical site was prepared by cleaning and sterilizing with iodine followed by 70% ethanol. A sterile surgical drape was used to drape the surgical site.

### Full-Thickness Wound Creation

In total 16 wounds (2 cm in diameter) were introduced on the back of each pig via surgical incision down to muscular layer. The incisions in a 4×4 pattern were approximately 4 cm apart of each other on either side of the dorsal back of the animal.

### Hydrogels as Wound Dressings for Micropigs Full Thickness Wound

Two different ultrashort peptide nanogels IVFK and IVZK and their variations that contained silver nanoparticles, IVFK-AgNPs and IVZK-AgNPs, were chosen in this study. Hydrogel-based dressings known as Duoderm Hydroactive Gel® and Aquacel® Hydrofiber AG, were chosen as controls. All dressings were topically applied and were slightly extended over the wound by approximately 1 cm. This guaranteed full coverage of the entire wound in a randomized pattern. Then, waterproof adhesive films, Tegaderm (3M Singapore), were used to secure the dressings with further protected by a covering. Animals were housed individually to avoid any disturbances to the wounds potentially caused by other pen mates. Wound dressings changes were done twice a week.

### Wound Healing Assessment

Wound contraction and wound closure were evaluated using digital planimetry. The tattooed margins were checked on days 4, 8, 11, 15, 18, 22, and 25 after initiation of the study. A photograph of the wound was taken every time wound change was conducted using amount at a fixed distance above the wound and a ruler as a scale adjacent to the wound and analyzed the data using image J software. The general linear model for the determination of time versus wound closure (re-epithelialization) and granulation tissue formation regarding each treatment was evaluated. The percentage of wound closure was considered by the

$$Wound closure ( \% ) = \frac{area at biopsy}{the area on incision day} \times 100 \qquad formula$$

and the percentage of wound area was calculated by applying the following equation:

$$Wound closure ( \% ) = \frac{wound area at day 0 - wound is at day x}{the area on wound area at day 0} \times 100$$

### Terminal Procedure (Animal Euthanasia)

The animals were anesthetized with isoflurane. They were sacrificed by exsanguination of the vena cava and aorta.

### Statistical Analysis

Three independent experiments were done for each type of test. A one-way analysis of the variance was used to determine the statistical differences between the experimental groups. Regarding the wound closure and re-epithelialization data, the wound area over observation time was considered. All results are presented as a mean±SD. Results are statistically significant when the p-values were P<0.05.

### REFERENCES

The following references are referred to above and are incorporated herein by reference:
1. Loo Y, Wong Y C, Cai E Z, Ang C H, Raju A, et al. (2014) Ultrashort peptide nanofibrous hydrogels for the acceleration of healing of burn wounds. Biomaterials 35: 4805-4814.
2. Pham C, Greenwood J, Cleland H, Woodruff P, Maddern G (2007) Bioengineered skin substitutes for the management of burns: a systematic review. Burns 33: 946-957. [Crossref]
3. CFD Center for Disease Control and Prevention (2014) National diabetes statistics report: estimates of diabetes and its burden in the United States, Atlanta, GA: U S Department of Health and Human Services 2014.
4. Fox L T, Mazumder A, Dwivedi A, Gerber M, du Plessis J (2017) In vitro wound healing and cytotoxic activity of the gel and whole-leaf materials from selected aloe species. J Ethnopharmacol 200: 1-7. [Crossref]
5. Langer R, Vacanti J P (1993) Tissue engineering. Science 260: 920-926. [Crossref]
6. Wassermann D, Schlotterer M, Toulon A, Cazalet C, Marien M, et al. (1988) Preliminary clinical studies of a biological skin equivalent in burned patients. Burns 14: 326-330.

7 Eaglstein W H, Falanga V (1997) Tissue engineering and the development of Apligraf, a human skin equivalent. Clin Ther 19: 894-905. [Crossref]
8. Rogers L C, Bevilacqua N J, Armstrong D G, Andros G (2010) Digital planimetry results in more accurate wound measurements: a comparison to standard ruler measurements, SAGE Publications.
9. Gao M, Nguyen T T, Suckow M A, Wolter W R, Gooyit M, et al. (2015) Acceleration of diabetic wound healing using a novel protease—anti-protease combination therapy. Proceedings of the National Academy of Sciences 112: 15226-15231.
10. Snoek-van beurden P A, Von den Hoff J W (2005) Zymographic techniques for the analysis of matrix metalloproteinases and their inhibitors. Biotechniques 38: 73-83.
11. Dai N T, Williamson M R, Khammo N, Adams E F, Coombes A G (2004) Composite cell support membranes based on collagen and polycaprolactone for tissue engineering of skin. Biomaterials 25: 4263-4271.
12. Li H, Berthod F, Xu W, Damour O, Germain L (1997) Use of in vitro reconstructed skin to cover skin flap donor site. J Surg Res 73: 143-148. [Crossref]
13. Wolcott R, Sanford N, Gabrilska R, Oates J L, Wilkinson J E (2016) Microbiota is a primary cause of pathogenesis of chronic wounds. J Wound Care 25: S33-S43. [Crossref]
14. McCloskey A P, Gilmore B F, Laverty G (2014) Evolution of antimicrobial peptides to self-assembled peptides for biomaterial applications. Pathogens 3: 791-821.
15. Gil E S, Panilaitis B, Bellas E, Kaplan D L (2013) Functionalized silk biomaterials for wound healing. Adv Healthc Mater 2: 206-217.
16. Choi J S, Leong K W, Yoo H S (2008) In vivo wound healing of diabetic ulcers using electrospun nanofibers immobilized with human epidermal growth factor (EGF). Biomaterials 29: 587-596.
17. Rice J J, Martino M M, De Laporte L, Tortelli F, Briquez P S, et al. (2013) Engineering the regenerative microenvironment with biomaterials. Adv Healthc Mater 2: 57-71.
18. Seow W Y, Hauser C A (2014) Short to ultrashort peptide nanogels for biomedical uses. Mater Today 17: 381-388.
19. Wu E C, Zhang S, Hauser C A (2012) Self-assembling peptides as cell-interactive scaffolds. Adv Functional Mater 22: 456-468.
20. Hauser C A E, Deng R, Mishra A, Loo Y, Khoe U, et al. (2011) Natural tri-to hexapeptides self-assemble in water to amyloid β-type fiber aggregates by unexpected a-helical intermediate structures. Proceedings of the National Academy of Sciences 108: 1361-1366.
21. Mishra A, Loo Y, Deng R, Chuah Y J, Hee H T, J Y Ying, et al. (2011) Ultrasmall natural peptides self-assemble to strong temperature-resistant helical fibers in scaffolds suitable for tissue engineering. Nano Today 6: 232-239.
22. Rauf S, Kahin K, Alshehri S, Abdelrahman S, Susapto H H, et al. (2018) In situ 3D bioprinting under physiological pH using ultrashort self-assembling peptides. (submitted).
23. Reithofer M R, Lakshmanan A, Ping A T, Chin J M, Hauser C A E (2014) In situ synthesis of size-controlled, stable silver nanoparticles within ultrashort peptide nanogels and their anti-bacterial properties. Biomaterials 35: 7535-7542.
24. Arab W, Rauf S, Al-Harbi O, Hauser C A E (2018) Novel Ultrashort Self-Assembling Peptide Bioinks for 3D Culture of Muscle Myoblast Cells. Int J Bioprinting 4: 1-13.
25. Loo Y, Lakshmanan A, Ni M, Toh L L, Wang S, et al. (2015) Peptide bioink: self-assembling nanofibrous scaffolds for three-dimensional organotypic cultures. Nano Letters 15: 6919-6925.
26. Ellis-Behnke R G, Liang Y X, You S W, Tay D K, Zhang S, et al. (2006) Nano neuro knitting: Peptide nanofiber scaffold for brain repair and axon regeneration with functional return of vision. Proceedings of the National Academy of Sciences 103: 5054-5059.
27. Gurtner G C, Werner S, Barrandon Y, Longaker M T (2008) Wound repair and regeneration. Nature 453: 314-321. [Crossref]
28. Sun G, Zhang X, Shen Y I, Sebastian R, Dickinson L E, et al. (2011) Dextran hydrogel scaffolds enhance angiogenic responses and promote complete skin regeneration during burn wound healing. Proceedings of the National Academy of Sciences 108: 20976-20981.
29. Place E S, Evans N D, Stevens M M (2009) Complexity in biomaterials for tissue engineering. Nat Mater 8: 457-470. [Crossref]
30. Jayawarna V, Ali M, Jowitt T A, Miller A F, Saiani A, et al. (2006) Nanostructured hydrogels for three-dimensional cell culture through self-assembly of fluorenylmethoxycarbonyl—dipeptides. Adv Mater 18: 611-614.
31. Dunn C J, Goa K L (1999) Tranexamic acid: a review of its use in surgery and other indications. Drugs 57: 1005-1032. [Crossref]
32. Martinez-Gutierrez F, Olive P L, Banuelos A, Orrantia E, Nino N, et al. (2010) Synthesis, characterization, and evaluation of antimicrobial and cytotoxic effect of silver and titanium nanoparticles. Nanomedicine 6: 681-688.
33. Lakshmanan A, Cheong D W, Accardo A, Di Fabrizio E, Riekel C, et al. (2013) Aliphatic peptides show similar self-assembly to amyloid core sequences, challenging the importance of aromatic interactions in amyloidosis. Proceedings of the National Academy of Sciences 110: 519-524.
34. Greenfield M A, Hoffman J R, de la Cruz M O, Stupp S I (2010) Tunable mechanics of peptide nanofiber gels. Langmuir 26: 3641-3647. [Crossref]
35. Mezger T G, The rheology handbook: for users of rotational and oscillatory rheometers, Vincentz Network GmbH & Co KG 2006.
36. Dominguez R, Holmes K C (2011) Actin structure and function. Annu Rev Biophys 40: 169-186. [Crossref]
37. Boucard N, Viton C, Agay D, Mari E, Roger T, et al. (2007) The use of physical hydrogels of chitosan for skin regeneration following third-degree burns. Biomaterials 28: 3478-3488.
38. Sullivan T P, Eaglstein W H, Davis S C, Mertz P (2001) The pig as a model for human wound healing. Wound Repair Regen 9: 66-76. [Crossref]
39. Velander P, Theopold C, Hirsch T, Bleiziffer O, Zuhaili B, et al. (2008) Impaired wound healing in an acute diabetic pig model and the effects of local hyperglycemia. Wound Repair Regen 16: 288-293.
40. Losi P, Briganti E, Errico C, Lisella A, Sanguinetti E, et al. Fibrin-based scaffold incorporating VEGF-and bFGF-loaded nanoparticles stimulates wound healing in diabetic mice, Acta Biomaterialia 9 (2013) 7814-7821
41. Yanagibayashi S, Kishimoto S, Ishihara M, Murakami K, Aoki H, et al. (2012) Novel hydrocolloid-sheet as wound dressing to stimulate healing-impaired wound healing in diabetic db/db mice. Biomed Mater Eng 22: 301-310.
42. Bielefeld K A, Amini-Nik S, Alman B A (2013) Cutaneous wound healing: recruiting developmental pathways for regeneration. Cell Mol Life Sci 70: 2059-2081.
43. Selig H F, Lumenta D B, Giretzlehner M, Jeschke M G, Upton D, et al. (2012) The properties of an “ideal” burn wound dressing—what do we need in daily clinical practice? Results of a worldwide online survey among burn care specialists. Burns 38: 960-966.
44. Cameron P, Gaiser B K, Bhandari B, Bartley P M, Katzer F, et al. (2016) Silver nanoparticles decrease the viability of Cryptosporidium parvum oocysts. Appl Environ Microbiol 82: 431-437.
45. Carrejo N C, Moore A N, Lopez Silva T L, Leach D G, Li I C, et al. (2018) Multidomain Peptide Hydrogel Accelerates Healing of Full-Thickness Wounds in Diabetic Mice. ACS Biomater Sci Eng 4: 1386-1396.

All documents, patents, journal articles and other materials cited in the present application are incorporated herein by reference.

While the present invention has been disclosed with references to certain embodiments, numerous modification, alterations, and changes to the described embodiments are possible without departing from the sphere and scope of the present invention, as defined in the appended claims. Accordingly, it is intended that the present invention is not limited to the described embodiments, but that it has the full scope defined by the language of the following claims, and equivalents thereof.

1. A peptide capable of forming a gel by self-assembly comprising:
at least one peptide selected from a group of peptides having a formula selected from AₙBₘX, BₘAₙX, XAₙBₘ, and XBₘAₙ,
wherein A is an aliphatic amino acid;
wherein B is comprised of at least one aromatic amino acid selected from the group consisting of: tyrosine, tryptophan, phenylalanine, and L-DOPA;
wherein X is comprised of a polar amino acid; and
wherein n being an integer being selected from 0-5 and m being an integer being selected from 0-3.

2. The peptide of claim 1, wherein amino acids in the peptide are either L-amino acids or D-amino acids.

3. The peptide of claim 1, wherein the at least one peptide selected from the group of peptides is selected from the group consisting of: IVFK and IVZK.

4. A hydrogel or organogel comprising a peptide, wherein the peptide comprises:
at least one peptide selected from a group of peptides having a formula selected from AₙBₘX, BₘAₙX, XAₙBₘ, and XBₘAₙ,
wherein A is an aliphatic amino acid;
wherein B is comprised of at least one aromatic amino acid selected from the group consisting of: tyrosine, tryptophan, phenylalanine, and L-DOPA;
wherein X is comprised of a polar amino acid; and
wherein n being an integer being selected from 0-5 and m being an integer being selected from 0-3.

5. The hydrogel or organogel of claim 4, wherein the hydrogel or organogel is comprised in at least one of a fuel cell, a solar cell, an electronic cell, a biosensing device, a medical device, an implant, a pharmaceutical composition and a cosmetic composition.

6. The hydrogel or organogel of claim 4, wherein N-terminus of the peptide is modified with a functional group selected from the group consisting of carboxylic acid, amide, alcohol, aldehyde, amine, imine, nitrile, an urea analog, phosphate, carbonate, sulfate, nitrate, maleimide, vinyl sulfone, azide, alkyne, alkene, carbohydrate, imide, peroxide, ester, aryl, ketone, sulphite, nitrite, phosphonate, and silane.

7. The hydrogel or organogel of claim 4, wherein C-terminus of the peptide is functionalized, by chemical conjugation or coupling of at least one compound selected from bioactive molecules or moieties, wherein the bioactive molecules or moieties are selected from the group consisting of growth factors, cytokines, lipids, cell receptor ligands, hormones, prodrugs, drugs, vitamins, antigens, antibodies, antibody fragments, oligonucleotides, and saccharides, and
wherein the chemical conjugation can be carried out before or after self-assembly of the peptide, peptidomimetic, or peptide and peptidomimetic.

8. The hydrogel or organogel of claim 4, wherein the peptide is employed in at least one of the group consisting of a medical tool kit, a fuel cell, a solar cell, an electronic cell, regenerative medicine and tissue regeneration, implantable scaffold disease model wound healing, 2D and 3D synthetic cell culture substrate, stem cell therapy, injectable therapies, biosensor development, high-throughput screening, biofunctionalized surfaces, printing biofabrication, bio-printing, and gene therapy.

9. A method of preparing a hydrogel or organogel, the method comprising:
dissolving a peptide in an aqueous solution or an organic solution, respectively, wherein the peptide comprises:
at least one peptide selected from a group of peptides having a formula selected from AₙBₘX, BₘAₙX, XAₙBₘ, and XBₘAₙ,
wherein A is an aliphatic amino acid;
wherein B is comprised of at least one aromatic amino acid selected from the group consisting of: tyrosine, tryptophan, phenylalanine, and L-DOPA;
wherein X is comprised of a polar amino acid; and
wherein n being an integer being selected from 0-5 and m being an integer being selected from 0-3.

10. The method of claim 9, wherein amino acids in the peptide are either L-amino acids or D-amino acids.

11. The method of claim 9, wherein the at least one peptide selected from the group of peptides is selected from the group consisting of: IVFK and IVZK.

12. The method of claim 9, wherein N-terminus of the peptide is modified with a functional group selected from the group consisting of carboxylic acid, amide, alcohol, aldehyde, amine, imine, nitrile, an urea analog, phosphate, carbonate, sulfate, nitrate, maleimide, vinyl sulfone, azide, alkyne, alkene, carbohydrate, imide, peroxide, ester, aryl, ketone, sulphite, nitrite, phosphonate, and silane.

13. The method of claim 9, wherein C-terminus of the peptide is functionalized, by chemical conjugation or coupling of at least one compound selected from bioactive molecules or moieties, wherein the bioactive molecules or moieties are selected from the group consisting of growth factors, cytokines, lipids, cell receptor ligands, hormones, prodrugs, drugs, vitamins, antigens, antibodies, antibody fragments, oligonucleotides, and saccharides, and wherein the chemical conjugation can be carried out before or after self-assembly of the peptide, peptidomimetic, or peptide and peptidomimetic.

14. The method of claim 9, wherein the peptide is employed in at least one of the group consisting of a medical tool kit, a fuel cell, a solar cell, an electronic cell, regenerative medicine and tissue regeneration, implantable scaffold disease model wound healing, 2D and 3D synthetic cell culture substrate, stem cell therapy, injectable therapies, biosensor development, high-throughput screening, biofunctionalized surfaces, printing biofabrication, bio-printing, and gene therapy.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. The method of claim 61, wherein the device is selected from the group consisting of a container with a dropper/closure device, a squeeze bottle pump spray, an airless and preservative-free spray, and an injectable device.

58. The method of claim 49, wherein the hydrogel or organogel is administered to a subject.

59. The method of claim 58, wherein the subject is a human.

60. The method of claim 58, wherein the subject is selected from the group consisting of a mammal, a reptile, a bird, a fish, an amphibian, and an invertebrate.

61. The method of claim 9, wherein the hydrogel or organogel is administered via a device.

62. The peptide of claim 1, further further comprising a pharmaceutically active compound.
